SEQUENCE LISTING (9 sequences)

SEQ ID NO: 1 - protein, 323 aa, cow
GEVASVPLTNYLDSQYFGKIYLGTPPQEFTVLFDTGSSDFWVPSIYCKSNACKNHQRFDPRKSSTFQNLGKPLSIHYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSVTISGVVVACEGGCQAILDTGTSKLVGPSSDILNIQQAIGATQNQYGEFDIDCDNLSYMPTVVFEINGKMYPLTPSAYTSQDQGFCTSGFQSENHSQKWILGDVFIREYYSVFDRANNLVGLAKAI

SEQ ID NO: 2 - protein, 323 aa, Buffalo
GEVASVPLTNYLDSQYFGKIYLGTPPQEFTVLFDTGSSDFWVPSIYCKSNACKNHQRFDPRKSSTFQNLGKPLSIRYGTGSMQGILGYDTVTVSNIVDIQQTVGLSTQEPGDVFTYAEFDGILGMAYPSLASEYSIPVFDNMMNRHLVAQDLFSVYMDRNGQESMLTLGAIDPSYYTGSLHWVPVTVQQYWQFTVDSITISGVVVACEGGCQAILDTGTSKLVGPSSDILNIQQAIGATQNQYGEFDIDCDNLSYMPTVVFEINGKMYPLTPSAYTSQDQGFCTSGFQSENRSQQWILGDVFIREYYSVFDRANNLVGLAKAI

SEQ ID NO: 3 - protein, 323 aa, Goat
GEVASVPLTNYLDSQYFGKIYLGTPPQEFTVLFDTGSSDFWVPSIYCKSNACKNHQRFDPRKSSTFQNLGKPLSIRYGTGSMQGILGYDTVTVSNIVDTQQTVGLSTQEPGDVFTYAEFDGILGMAYPSLASEYSVPVFDNMMDRRLVAQDLFSVYMDRNGQGSMLTLGAIDPSYYTGSLHWVPVTLQKYWQFTVDSVTISGAVVACEGGCQAILDTGTSKLVGPSSDILNIQQAIGATQNQYGEFDIDCDSLSSMPTVVFEINGKMYPLTPYAYTSQEEGFCTSGFQGENHSHQWILGDVFIREYYSVFDRANNLVGLAKAI

SEQ ID NO: 4 - protein, 323 aa, Sheep
GEVASVPLTNYLDSQYFGKIYLGTPPQEFTVLFDTGSSDFWVPSIYCKSNACKNHQRFDPRKSSTFQNLGKPLSIRYGTGSMQGILGYDTVTVSNIVDIQQTVGLSTQEPGDVFTYAEFDGILGMAYPSLASEYSVPVFDNMMDRRLVAQDLFSVYMDRSGQGSMLTLGAIDPSYYTGSLHWVPVTLQKYWQFTVDSVTISGAVVACEGGCQAILDTGTSKLVGPSSDILNIQQAIGATQNQYGEFDIDCDSLSSMPTVVFEINGKMYPLTPYAYTSQEEGFCTSGFQGENHSHQWILGDVFIREYYSVFDRANNLVGLAKAI

SEQ ID NO: 5 - protein, 323 aa, Camel [sequence omitted]

SEQ ID NO: 6 - protein, 323 aa, Pig
GEVASEPLTNYLDTQYFGKIYIGTPPQEFTVVFDTGSSELWVPSVYCKSDACQNHHRFNPSKSSTFQNLDKPLSIQYGTGSIQGFLGYDTVMVAGIVDAHQTVGLSTQEPSDIFTYSEFDGILGLGYPELASEYTVPVFDNMMHRHLVAQDLFAVYMSRNDEGSMLTLGAIDPSYYTGSLHWVPVTMQLYWQFTVDSVTINGVVVACNGGCQAILDTGTSMLAGPSSDILNIQMAIGATESQYGEFDIDCGSLSSMPTVVFEISGRMYPLPPSAYTNQDQGFCTSGFQGDSKSQHWILGVVFIQEYYSVFDRANNRVGLAKAI

SEQ ID NO: 7 - protein, 330 aa, Cow
AATLVSEQPLQNYLDTEYFGTIGIGTPAQDFTVIFDTGSSNLWVPSIYCSSEACTNHNRFNPQDSSTYEATSETLSITYGTGSMTGILGYDTVQVGGISDTNQIFGLSETEPGSFLYYAPFDGILGLAYPSISSSGATPVFDNIWDQGLVSQDLFSVYLSSNEESGSVVIFGDIDSSYYSGSLNWVPVSVEGYWQITVDSITMNGESIACSDGCQAIVDTGTSLLAGPTTAISNIQSYIGASEDSSGEVVISCSSIDSLPDIVFTINGVQYPVPPSAYILQSNGICSSGFEGMDISTSSGDLWILGDVFIRQYFTVFDRGNNQIGLAPVA

SEQ ID NO: 8 - protein, 361 aa, Mucor
AAADGSVDTPGYYDFDLEEYAIPVSIGTPGQDFLLLFDTGSSDTWVPHKGCTKSEGCVGSRFFDPSASSTFKATNYNLNITYGTGGANGLYFEDSIAIGDITVTKQILAYVDNVRGPTAEQSPNADIFLDGLFGAAYPDNTAMEAEYGSTYNTVHVNLYKQGLISSPLFSVYMNTNSGTGEVVFGGVNNTLLGGDIAYTDVMSRYGGYYFWDAPVTGITVDGSAAVRFSRPQAFTIDTGTNFFIMPSSAASKIVKAALPDATETQQGWVVPCASYQNSKSTISIVMQKSGSSSDTIEISVPVSKMLLPVDQSNETCMFIILPDGGNQYIVGNLFLRFFVNVYDFGNNRIGFAPLASAYENE

SEQ ID NO: 9 - protein, 330 aa, Endotheia
STGSATTTPIDSLDDAYITPVQIGTPAQTLNLDFDTGSSDLWVFSSETTASEVDGQTIYTPSKSTTAKLLSGATWSISYGDGSSSSGDVYTDTVSVGGLTVTGQAVESAKKVSSSFTEDSTIDGLLGLAFSTLNTVSPTQQKTFFDNAKASLDSPVFTADLGYHAPGTYNFGFIDTTAYTGSITYTAVSTKQGFWEWTSTGYAVGSGTFKSTSIDGIADTGTTLLYLPATVVSAYWAQVSGAKSSSSVGGYVFPCSATLPSFTFGVGSARIVIPGDYIDFGPISTGSSSCFGGIQSSAGIGINIFGDVALKAAFVVFNGATTPTLGFASK

(19) United States
(12) Patent Application Publication
(10) Pub. No.: US 2017/0202230 A1
(43) Pub. Date: Jul. 20, 2017

(54) MILK CLOTTING ASPARTIC PROTEASE ENZYME COMPOSITION

(71) Applicant: Chr. Hansen A/S, Hoersholm (DK)

(72) Inventors: MARTIN LUND, Copenhagen (DK); Enikoe Fodor HANSEN, Helsingoer (DK); Jonas JACOBSEN, Copenhagen (DK); Johannes Maarten VAN DEN BRINK, Herlev (DK)

(73) Assignee: Chr. Hansen A/S, Hoersholm (DK)

(21) Appl. No.: 15/324,865

(22) PCT Filed: Jul. 10, 2015
(86) PCT No.: PCT/EP2015/065885

§ 371 Date: Jan. 9, 2017

(30) Foreign Application Priority Data
Jul. 11, 2014 (EP) 14176744.2

(51) Int. Cl. A23C 9/12; A23L 33/10; A23K 20/189; C12N 9/64; C12N 9/08

(52) U.S. Cl. CPC A23C 9/1209; C12N 9/6483; C12N 9/6481; C12Y 304/23023; C12Y 304/23004; C12Y 111/01007; C12N 9/0065; A23K 20/189; A23L 33/10

(57) ABSTRACT

A liquid or dried granulated milk clotting aspartic protease enzyme composition comprising added polypeptides/proteins. The polypeptides/proteins may be animal-derived (e.g. whey, lactalbumin, transferrin, casein, ovalbumin, gelatin, blood), vegetable-derived (soy, pea, corn, potato, hemp, rice, wheat, peanut, sun flower, rape seed) or algae proteins (e.g. spirulina). Addition of protein in several instances increases activity of the enzyme and simultaneously improves physical stability.

### FIELD OF THE INVENTION

The present invention relates to liquid or dried granulated milk clotting aspartic protease enzyme composition comprising added polypeptides/proteins.

### BACKGROUND ART

Enzymatic coagulation of milk by milk-clotting enzymes, such as chymosin and pepsin, is one of the most important processes in the manufacture of cheeses. Enzymatic milk coagulation is a two-phase process: a first phase where a proteolytic enzyme, chymosin or pepsin, attacks κ-casein, resulting in a metastable state of the casein micelle structure and a second phase, where the milk subsequently coagulates and forms a coagulum.

Chymosin (EC 3.4.23.4) and pepsin (EC 3.4.23.1), the milk clotting enzymes of the mammalian stomach, are aspartic proteases belonging to a broad class of peptidases.

Mucorpepsin (EC 3.4.23.23) is a milk clotting enzyme derived from the fungus Rhizomucor miehei.

Commercial relevant milk-clotting enzyme products are often liquid compositions and in the art is described numerous different ways to try to stabilize the milk-clotting enzyme in the product—e.g. to improve storage stability or specific activity of the enzyme.

For instance, EP2333056A1 (DSM, date of filing Dec. 4, 2007) describes that formate, acetate, lactate, propionate, malate, fumarate or propanediol may increase stability of aspartic protease enzyme in a liquid composition/product.

WO2012/127005A1 (DSM) describes a stable liquid chymosin composition comprising inorganic salt in a concentration of 2-100 g/kg and a preservative such as formate, acetate, lactate, propionate, malate, benzoate, sorbate or fumarate, glycol (ethanediol), propylene glycol (propanediol), glycerol, erythritol, xylitol, mannitol, sorbitol, inositol or galactitol. The highest strength of the described chymosin compositions is1500 IMCU/ml (see e.g. page 6, lines1-3).

Polyethylene glycol (PEG) is a polymer of ethylene oxide—it may alternatively be termed polyoxyethylene (POE). PEG is commercially available over a wide range of molecular weights such as from 300 g/mol to 10,000,000 g/mol.

DE1492060A1 (Nordmark-Werke GmbH, published in 1969) discloses a method for making a pepsin composition by adding Polyethylene glycol (PEG) with a molecular weight of 400-6000 at a concentration of 1-20 wt % (corresponds to 10,000 to 200,000 ppm).

### SUMMARY OF THE INVENTION

A problem to be solved by the present invention is to provide a novel milk clotting aspartic protease enzyme (e.g. chymosin) composition, wherein the aspartic protease has increased physical stability and/or specific activity.

The solution is based on that the present inventors have identified that by adding suitable polypeptide/protein formulations to different aspartic protease enzymes (e.g. chymosin) one significantly improves the physical stability and the specific activity of the enzyme compositions.

Liquid formulations of industrial enzymes are subjected to physical forces (such as shaking) during e.g. transportation and physical stability of an enzyme composition can be tested by repeatable shaking (e.g. via inversion) a sample in a test tube having high head space to sample volume ratio.

As discussed in working Examples herein—to e.g. camel chymosin (CHY-MAX® M, Chr. Hansen A/S) were added numerous different polypeptide/protein formulations and a number of these polypeptide/protein formulations significantly increased the physical stability of camel chymosin.

As discussed in working Examples herein—to bovine chymosin (CHY-MAX®, Chr. Hansen A/S), camel chymosin (CHY-MAX® M, Chr. Hansen A/S) and mucorpepsin (Hannilase®, Chr. Hansen A/S) were added numerous different polypeptide/protein formulations and a number of these polypeptide/protein formulations significantly increased the specific activity of the enzyme compositions.

The increase in the specific activity was most significant for the bovine and camel chymosins.

As understood by the skilled person in the present context—specific activity of a milk clotting aspartic protease enzyme composition relates to activity/IMCU per total amount of milk clotting aspartic protease enzyme protein in the composition.

As described in working Examples herein—addition of e.g. whey protein formulations to a purified chymosin sample gave an enzyme composition with significant higher IMCU/ml strength—i.e. a composition with higher specific activity.

For instance, addition of 0.5% (w/w) of whey protein concentrate formulation WPC 80 gave around 10% increase of the strength in the camel chymosin composition as such.

Without being limited to theory—it was a surprise to the present inventors that it was possible to increase the strength of e.g. a chymosin composition/product by simply adding a suitable amount of e.g. whey proteins.

It is here relevant to note that casein hydrolysate did not significantly increase the physical stability and/or the specific activity.

As known to the skilled person—in a casein hydrolysate has been performed a hydrolysis of casein.

Without being limited to theory—it is believed that in such a casein hydrolysate formulation a significant amount of the polypeptides are of less than 10 amino acids.

Said in other words and without being limited to theory—it is believed that the herein relevant increased physical stability and specific activity effects are obtained when there are used polypeptides longer than 10 amino acids.

As known in the art—the term peptide may be distinguished from the term protein on the basis of size, which as an arbitrary benchmark may be understood to be approximately 50 or fewer amino acids.

Said in other words, a polypeptide longer than 50 amino acids may normally in the art be understood to be a protein.

Accordingly, a polypeptide longer than 50 amino acids may herein alternatively be termed a protein.

As discussed in working Examples herein—the herein relevant increased/improved physical stability and/or specific activity effects were related to the amount of polypeptide/protein composition added to chymosin—where no significant positive effect was obtained if less than 0.01% (w/w) was added.

Without being limited to theory—it is believed that addition of polypeptide/protein provide increased conformational stability to the chymosins and this could explain the observed increased physical stability and increased specific activity observed in working Examples herein.

Without being limited to theory—it is believed that in the prior art it has not been described or suggested that addition of polypeptide/protein may increase the stability of aspartic protease milk-clotting enzymes such as e.g. chymosins—in particular it has not been described that conformational stability may be increased.

Conformational stability of an enzyme is illustrated in FIG. 3 herein.

As known in the art—loss of conformation equals loss of activity of the enzyme—i.e. less specific activity of the enzyme.

Without being limited to theory—loss of conformation may increase denaturation/precipitation of the enzyme and thereby give less physical stability as discussed herein.

As known in the art—milk clotting aspartic protease enzymes may be seen as structurally relatively similar.

As known in the art—different natural wildtype milk clotting aspartic protease polypeptide sequences obtained from different mammalian or fungal species (such as e.g. bovines, camels, sheep, pigs, or mucor) are having a relatively high tertiary structural similarity.

In FIG. 4 herein this is provided an alignment of herein relevant different milk clotting chymosin sequences from different mammalian species (cow, buffalo, goat, sheep, camel and pig)—as can be seen in FIG. 4 they have a close sequence relationship and are known to have a very high tertiary structural similarity.

In FIG. 5 herein there is provided an alignment of herein relevant commercially available different milk clotting aspartic protease enzymes sequences from different mammalian or fungal species (camel chymosin, cow chymosin, cow pepsin, fungal mucor pepsin and fungal Endothia pepsin).

It may be said that the 5 different sequences of FIG. 5 are not highly identical—but as known to the skilled person all these 5 different milk clotting aspartic protease enzymes are known to have a high tertiary structural similarity.

As discussed above and shown in working Examples herein—the herein relevant improved/increased stability/activity effects have been demonstrated for bovine chymosin, camel chymosin and mucorpepsin.

Without being limited to theory—it is believed that there is no significant technical reason to believe that the herein relevant improved/increased stability effect should not be relevant for milk clotting aspartic protease enzymes in general—as discussed above, they are known to have a high tertiary structural similarity and as understood by the skilled person in the present context this tertiary structural similarity makes it plausible that the herein described polymer-enzyme interaction to get improved stability would be a general class effect of the structural similar herein relevant milk clotting aspartic protease enzymes.

Accordingly, a first aspect of the invention relates to a method for making a liquid milk clotting aspartic protease enzyme composition, wherein the method comprises the steps of:
(a): obtaining a purified liquid milk clotting aspartic protease enzyme sample comprising:
   (i): a strength of from 25 IMCU/g to 30,000 IMCU/g of the sample; and
   (ii): wherein at least 70% of the total amounts of proteins with a size bigger than 10 kDa, determined by SDS-PAGE, in the purified sample are milk clotting aspartic protease enzyme; and
(b): adding a suitable amount of a polypeptide formulation comprising polypeptides longer than 10 amino acids to the sample of step (a), wherein the polypeptides are not milk clotting aspartic protease enzymes and not enzymes that degrade the aspartic protease enzymes, to get a liquid milk clotting aspartic protease enzyme composition, wherein the composition comprises:
   (I): milk clotting aspartic protease enzyme at a strength of from 26 IMCU/g to 30,100 IMCU/g of the composition; and
   (II): the in step (b) added not milk clotting aspartic protease enzyme polypeptides longer than 10 amino acids in a concentration from 0.01% to 10% (w/w) of the composition; and
      wherein the strength (IMCU/g of the composition) in (I) is at least 1% higher than the strength in (i) (IMCU/g of the sample), measured after one week of storage at 5° C.

It is routine work to measure the milk clotting aspartic protease enzyme strength in (i) and in (II) and thereby identify if the requirement of higher strength in (II) is fulfilled. In the present context, it is evident to the skilled person that the control sample to measure the strength in (i) after one week of storage is the sample of step (a) (i.e. without the added polypeptide formulation of step (b)).

As discussed herein, numerous of polypeptide/protein formulations were tested by the present inventors (see working Examples herein) and a number of these significantly increased the specific activity of the enzyme compositions.

Accordingly, based on the teaching herein and the common general knowledge of the skilled person—the skilled person may without undue burden identify a suitable preferred protein formulation to be added in a suitable amount in step (b) in order get the required increased/higher strength of the method of the first aspect.

In the present context, it is evident that in step (b) of the method of the first aspect it is not preferred to add enzymes that degrade the aspartic protease enzymes and the term “not enzymes that degrade the aspartic protease enzymes” of step (b) should be understood in relation to this.

The concentration in item (II) relates to the composition as such.

For instance, if the weight of the composition as such is1 kg and the concentration in item (II) of the in step (b) added polypeptides is1% (w/w) then has there in step (b) been added 10 g polypeptides longer than 10 amino acids.

It is routine work for the skilled person to add a suitable amount of a polypeptide formulation in step (b) in order to get a wanted concentration of the added polypeptides in accordance with item (II) of the first aspect.

In FIG. 1 herein is shown SDS-PAGE data for different samples of bovine chymosin (CHY-MAX®, Chr. Hansen A/S) and mucorpepsin (Hannilase®, Chr. Hansen A/S). For both CHY-MAX® and Hannilase® are Lane 2 (named “Feed”) unpurified samples essentially taken from the fermentation media and Lane 3 are purified samples.

As can be seen—the purified samples are within the scope of item (ii) of the first aspect—i.e. they are samples, wherein at least 70% of the total amounts of proteins with a size bigger than 10 kDa are the milk clotting aspartic protease enzyme.

The not purified samples are not within the scope of item (ii) of the first aspect.

SDS-PAGE is a well-known technology for the skilled person—i.e. it is routine work for the skilled person to determine if purified milk clotting aspartic protease enzyme sample of interest is a sample with the scope of item (ii) of the first aspect.

In FIG. 1 herein can be seen that there are two bands in the migration range 34-38 kDa for e.g. CHY-MAX® in purified sample of lane 3.

Without being limited to theory—it is believed that these two bands represent two different glycosylated forms of CHY-MAX®.

As understood by the skilled person in the present context—both of these different glycosylated forms are milk clotting aspartic protease enzymes, since they both have milk-clotting enzymatic activity.

As understood by the skilled person in the present context—the term “IMCU/g of the sample” in item (i) relates to IMCU enzyme activity per gram of the sample as such.

Similar—the term “IMCU/g of the composition” in item (I) relates to IMCU enzyme activity per gram of the composition as such.

It may be preferred that the liquid composition of the first aspect has a total weight of from 10 g to 10,000 kg.

As known to the skilled person in the present context—a herein relevant liquid composition of the first aspect that has a weight of 1 kg will approximately have a volume of 1 liter.

A second aspect of the invention relates to a liquid milk clotting aspartic protease enzyme composition obtainable by a method of the first aspect or any herein relevant embodiments thereof.

The first and second aspects herein relate to a liquid composition—however, milk clotting aspartic protease enzymes (e.g. chymosin) may also be commercialized as dried granulated composition/product.

Accordingly, a third aspect of the invention relates to a method for making a dried milk clotting aspartic protease enzyme composition, wherein the method comprises the steps of:
(1): first making a liquid milk clotting aspartic protease enzyme composition according to the method of the first aspect or any herein relevant embodiments thereof;
(2): drying and granulating the liquid composition of step (1) to get the dried granulated milk clotting aspartic protease enzyme composition.

The drying step (2) may be seen as a routine step for the skilled person in the present context—it is therefore not necessary to describe this step as such in details herein.

A fourth aspect of the invention relates to a dried granulated milk clotting aspartic protease enzyme composition obtainable by a method of the third aspect or any herein relevant embodiments thereof.

A fifth aspect of the invention relates to a liquid milk clotting aspartic protease enzyme composition comprising:
(I): milk clotting aspartic protease enzyme at a strength of from 25 IMCU/g to 30,000 IMCU/g of the composition;
(II): not milk clotting aspartic protease enzyme polypeptides longer than 10 amino acids in a concentration from 0.01% to 10% (w/w) of the composition; and
(III): a salt in a concentration from 1 to 350 g/kg and wherein the pH of the composition is from 2 to 8; and
   (x): wherein the polypeptides longer than 10 amino acids of item (II) are at least one polypeptide selected from the group of polypeptides consisting of: whey proteins, alpha lactalbumin, beta-lactoglobulin, transferrin, lactoperoxidase, casein, alpha-s1-casein, alpha-s2-casein, beta-casein, kappa-casein, ovalbumin, gelatin, bovine serum albumin, soy proteins, pea proteins, corn proteins, potato proteins, hemp proteins, rice proteins, spirulina proteins, wheat proteins, peanut proteins, sun flower proteins, rape seed proteins, blood proteins and algae proteins.

In the present context—the skilled person will know or may routinely determine (e.g. based on specific relevant amino acid sequences) the origin of the polypeptides longer than 10 amino acids of item (x).

As understood by the skilled person in the present context—the term “g/kg” in relation to item (III) relates to gram salt per kg of the composition as such.

A sixth aspect of the invention relates to a dried granulated milk clotting aspartic protease enzyme composition comprising:
(I): milk clotting aspartic protease enzyme at a strength of from 25 IMCU/g to 30,000 IMCU/g of the composition;
(II): not milk clotting aspartic protease enzyme polypeptides longer than 10 amino acids in a concentration from 0.01% to 10% (w/w) of the composition; and
(III): a salt and wherein the pH of the composition suspended in water is from 2 to 8; and
   (x): wherein the polypeptides longer than 10 amino acids of item (II) are at least one polypeptide selected from the group of polypeptides consisting of: whey proteins, alpha lactalbumin, beta-lactoglobulin, transferrin, lactoperoxidase, casein, alpha-s1-casein, alpha-s2-casein, beta-casein, kappa-casein, ovalbumin, gelatin, bovine serum albumin, soy proteins, pea proteins, corn proteins, potato proteins, hemp proteins, rice proteins, spirulina proteins, wheat proteins, peanut proteins, sun flower proteins, rape seed proteins, blood proteins and algae proteins.

A seventh aspect of the invention relates to a liquid milk clotting aspartic protease enzyme composition comprising milk clotting aspartic protease enzyme at a strength of from 1600 IMCU/g to 30,000 IMCU/g of the composition and a salt in a concentration from 1 to 350 g/kg and wherein the pH of the composition is from 2 to 8.

An eight aspect of the invention relates to a dried granulated milk clotting aspartic protease enzyme composition comprising milk clotting aspartic protease enzyme at a strength of from 1600 IMCU/g to 30,000 IMCU/g of the composition and a salt and wherein the pH of the composition suspended in water is from 2 to 8.

A milk clotting aspartic protease enzyme composition as described herein may be used according to the art—e.g. to make a food or feed product of interest (such as e.g. a milk based product of interest that e.g. could be a cheese product).

Accordingly, a ninth aspect of the invention relates to a method for making a food or feed product comprising adding an effective amount of a milk clotting aspartic protease enzyme composition of any of fifth to eight aspect or any herein relevant embodiments thereof to the food or feed ingredient(s) and carrying out further manufacturing steps to obtain the food or feed product.

### Definitions

All definitions of herein relevant terms are in accordance of what would be understood by the skilled person in relation to the herein relevant technical context.

The term “milk-clotting enzyme” refers to an enzyme with milk-clotting enzymatic activity—i.e. an active milk-clotting enzyme. The milk-clotting activity may be expressed in International Milk-Clotting Units (IMCU) per ml or IMCU per g. The skilled person knows how to determine herein relevant milk-clotting enzymatic activity. In working Example 1 herein is provided an example of a standard method to determine milk-clotting enzymatic activity and specific milk-clotting enzymatic activity. As known in the art—specific clotting activity (IMCU/mg total protein) is determined by dividing the clotting activity (IMCU/ml) by the total protein content (mg total protein per ml).

The term “Sequence Identity” relates to the relatedness between two amino acid sequences.

For purposes of the present invention, the degree of sequence identity between two amino acid sequences is determined according to the art and preferably determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment).

The term “variant” means a peptide having milk-clotting enzymatic activity comprising an alteration, i.e., a substitution, insertion, and/or deletion, at one or more (several) positions. A substitution means a replacement of an amino acid occupying a position with a different amino acid; a deletion means removal of an amino acid occupying a position; and an insertion means adding 1-3 amino acids adjacent to an amino acid occupying a position.

The amino acid may be natural or unnatural amino acids—for instance, substitution with e.g. a particularly D-isomers (or D-forms) of e.g. D-alanine could theoretically be possible.

Embodiment of the present invention is described below, by way of examples only.

### DRAWINGS

FIG. 1: In FIG. 1 herein is shown SDS-PAGE data for different samples of bovine chymosin (CHY-MAX®, Chr. Hansen A/S) and mucorpepsin (Hannilase®, Chr. Hansen A/S). For both CHY-MAX® and Hannilase® are Lane 2 (named “Feed”) unpurified samples essentially taken from the fermentation media and Lane 3 are purified samples.

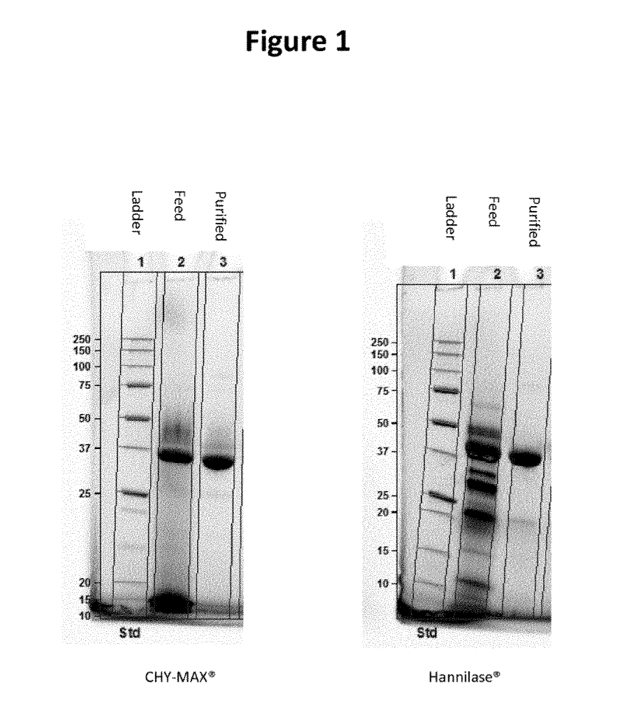

FIG. 2: Shows some of the results of inversion experiments as discussed herein to determine herein relevant physical stability. See working Example herein for further details.

FIG. 3: Conformational stability of an enzyme is illustrated.

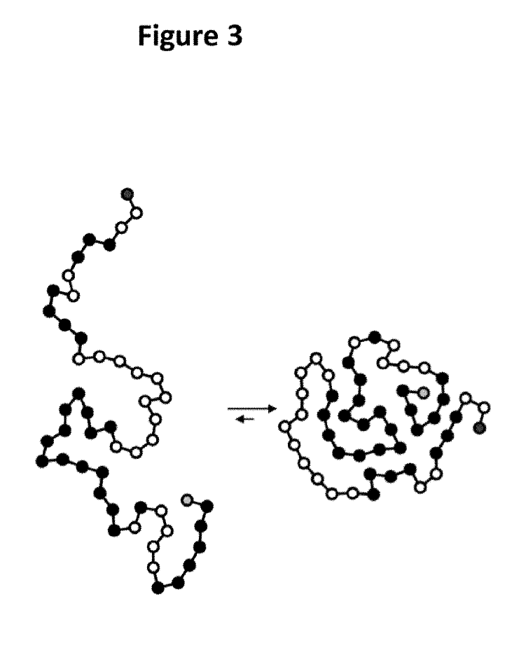

FIG. 4: An alignment of herein relevant different milk clotting chymosin sequences from different mammalian species (cow, buffalo, goat, sheep, camel and pig). All the sequences of FIG. 4 are public available.

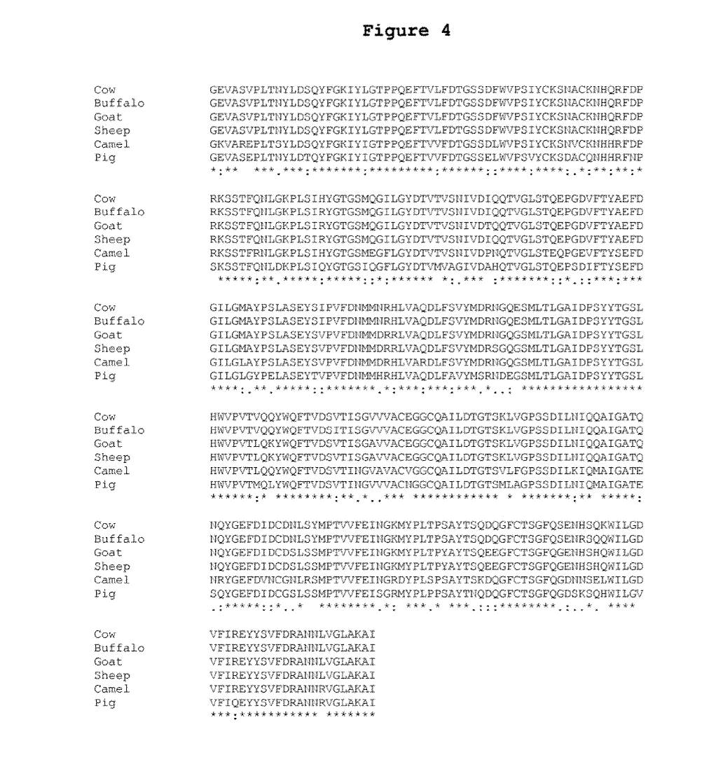

FIG. 5: An alignment of herein relevant commercially available different milk clotting aspartic protease enzymes sequences from different mammalian or fungal species (camel chymosin, cow chymosin, cow pepsin, fungal mucor pepsin and fungal Endothia pepsin). All the sequences of FIG. 5 are public available.

### DETAILED DESCRIPTION OF THE INVENTION

### Milk Clotting Aspartic Protease Enzyme

The discussion of specific embodiments/examples of herein relevant milk clotting aspartic protease enzymes below is relevant for all the aspects of the invention as discussed herein.

In a preferred embodiment, the milk clotting aspartic protease enzyme is a milk-clotting enzyme selected from the group consisting of chymosin (EC 3.4.23.4), pepsin (EC 3.4.23.1) and mucorpepsin (EC 3.4.23.23).

As discussed in working Examples herein—the herein relevant increase in the specific activity and strength were most significant for the bovine and camel chymosin compositions.

Accordingly, in a preferred embodiment the milk clotting aspartic protease enzyme is chymosin (EC 3.4.23.4).

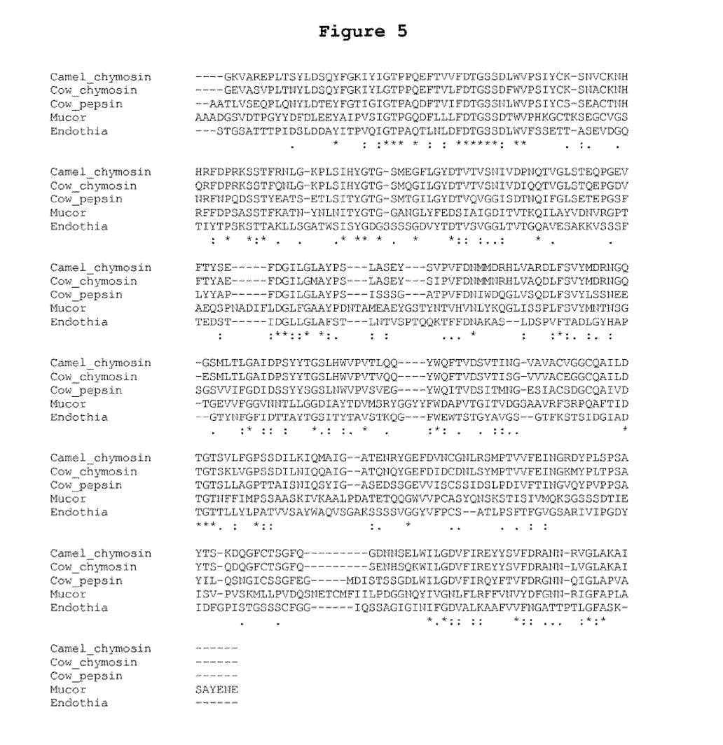

A preferred milk clotting aspartic protease enzyme is Camelius dromedarius chymosin as described in e.g. WO02/36752A2 (Chr. Hansen). It may herein alternatively be termed camel chymosin and the publically known mature polypeptide amino acid sequence is shown in FIG. 5 herein.

As known in the art—it is routine work for the skilled person to make variants (i.e. amino acid modifications) of an enzyme of interest without significantly changing the characteristics of the enzyme.

Accordingly, in a preferred embodiment the milk clotting aspartic protease enzyme is Camelius dromedarius chymosin comprising the polypeptide amino acid sequence shown in FIG. 5 herein (termed “Camel_chymosin”) or a variant of Camelius dromedarius chymosin, wherein the variant comprises a polypeptide sequence which has at least 90% (preferably at least 95%, more preferably at least 99%) sequence identity with the camel chymosin polypeptide amino acid sequence shown in FIG. 5 herein.

A preferred milk clotting aspartic protease enzyme is bovine chymosin. It may herein alternatively be termed cow chymosin and the publically known mature polypeptide amino acid sequence is shown in FIG. 5 herein.

Accordingly, in a preferred embodiment the milk clotting aspartic protease enzyme is bovine chymosin comprising the polypeptide amino acid sequence shown in FIG. 5 herein (termed “Cow_chymosin”) or a variant of bovine chymosin, wherein the variant comprises a polypeptide sequence which has at least 90% (preferably at least 95%, more preferably at least 99%) sequence identity with the bovine chymosin polypeptide amino acid sequence shown in FIG. 5 herein.

A preferred milk clotting aspartic protease enzyme is bovine pepsin. It may herein alternatively be termed cow pepsin and the publically known mature polypeptide amino acid sequence is shown in FIG. 5 herein.

Accordingly, in a preferred embodiment the milk clotting aspartic protease enzyme is bovine pepsin comprising the polypeptide amino acid sequence shown in FIG. 5 herein (termed “Cow_pepsin”) or a variant of bovine pepsin, wherein the variant comprises a polypeptide sequence which has at least 90% (preferably at least 95%, more preferably at least 99%) sequence identity with the bovine pepsin polypeptide amino acid sequence shown in FIG. 5 herein.

A preferred milk clotting aspartic protease enzyme is Mucor pepsin (see e.g. EP0805866B1 (Harboe et al, Chr. Hansen A/S, Denmark)). The publically known mature polypeptide amino acid sequence is shown in FIG. 5 herein.

Accordingly, in a preferred embodiment the milk clotting aspartic protease enzyme is Mucor pepsin comprising the polypeptide amino acid sequence shown in FIG. 5 herein (termed “Mucor”) or a variant of Mucor pepsin, wherein the variant comprises a polypeptide sequence which has at least 90% (preferably at least 95%, more preferably at least 99%) sequence identity with the Mucor pepsin polypeptide amino acid sequence shown in FIG. 5 herein.

A preferred milk clotting aspartic protease enzyme is Endothia pepsin. The publically known mature polypeptide amino acid sequence is shown in FIG. 5 herein.

Accordingly, in a preferred embodiment the milk clotting aspartic protease enzyme is Endothia pepsin comprising the polypeptide amino acid sequence shown in FIG. 5 herein (termed “Endothia”) or a variant of Endothia pepsin, wherein the variant comprises a polypeptide sequence which has at least 90% (preferably at least 95%, more preferably at least 99%) sequence identity with the Endothia pepsin polypeptide amino acid sequence shown in FIG. 5 herein.

### Added Polypeptide/Proteins

As discussed above—step (b) of the method of the first aspect reads:
step (b): adding a suitable amount of a polypeptide formulation comprising polypeptides longer than 10 amino acids to the sample of step (a), wherein the polypeptides are not milk clotting aspartic protease enzymes and not enzymes that degrade the aspartic protease enzymes, to get a liquid milk clotting aspartic protease enzyme composition,

As discussed above—item (II) of the liquid milk clotting aspartic protease enzyme composition of the fifth aspect and item (II) of the dried granulated milk clotting aspartic protease enzyme composition of the sixth aspect reads:
(II): not milk clotting aspartic protease enzyme polypeptides longer than 10 amino acids in a concentration from 0.01% to 10% (w/w) of the composition;

The discussion of specific embodiments/examples of herein relevant “polypeptides longer than 10 amino acids” below is relevant for all the aspects of the invention as discussed herein.

In the present context, it is evident that in step (b) of the method of the first aspect it is not preferred to add enzymes that degrade the aspartic protease enzymes and the term “not enzymes that degrade the aspartic protease enzymes” of step (b) should be understood in relation to this.

Preferably, the polypeptides longer than 10 amino acids are at least one polypeptide selected from the group of polypeptides consisting of: whey proteins, alpha lactalbumin, beta-lactoglobulin, transferrin, lactoperoxidase, casein, alpha-s1-casein, alpha-s2-casein, beta-casein, kappa-casein, ovalbumin, gelatin, bovine serum albumin, soy proteins, pea proteins, corn proteins, potato proteins, hemp proteins, rice proteins, spirulina proteins, wheat proteins, peanut proteins, sun flower proteins, rape seed proteins, blood proteins and algae proteins.

More preferably, the polypeptides longer than 10 amino acids are at least one polypeptide selected from the group of polypeptides consisting of: whey proteins, alpha lactalbumin, beta-lactoglobulin, transferrin, lactoperoxidase, casein, alpha-s1-casein, alpha-s2-casein, beta-casein, kappa-casein, ovalbumin, gelatin and bovine serum albumin.

Within the group immediately above it is preferred that the polypeptides longer than 10 amino acids are at least one polypeptide selected from the group of polypeptides consisting of: whey proteins, alpha lactalbumin, beta-lactoglobulin, casein, alpha-s1-casein, alpha-s2-casein, beta-casein, kappa-casein, ovalbumin, gelatin and bovine serum albumin.

In a preferred embodiment—polypeptides longer than 10 amino acids are polypeptides longer than 25 amino acids, more preferably polypeptides longer than 40 amino acids.

As discussed in working Examples herein—herein relevant positive results were obtained by addition of e.g. whey protein, ovalbumin and BSA, which herein all may be characterized as relatively large proteins.

As known in the art—the term peptide may be distinguished from the term protein on the basis of size, which as and as an arbitrary benchmark may be understood to be approximately 50 or fewer amino acids.

Said in other words, a polypeptide longer than 50 amino acids may normally in the art be understood to be a protein.

Accordingly, a polypeptide longer than 50 amino acids may herein alternatively be termed a protein.

In a preferred embodiment—polypeptides longer than 10 amino acids are proteins longer than 50 amino acids, more preferably proteins longer than 75 amino acids, even more preferably proteins longer than 150 amino acids.

It may even be preferred that polypeptides longer than 10 amino acids are proteins longer than 300 amino acids.

### First Aspect—A Method for Making a Liquid Milk Clotting Aspartic Protease Enzyme Composition

As discussed above—the first aspect of the invention relates to a method for making a liquid milk clotting aspartic protease enzyme composition, wherein the method comprises the steps of:
(a): obtaining a purified liquid milk clotting aspartic protease enzyme sample comprising:
   (i): a strength of from 25 IMCU/g to 30,000 IMCU/g of the sample; and
   (ii): wherein at least 70% of the total amounts of proteins with a size bigger than 10 kDa, determined by SDS-PAGE, in the purified sample are milk clotting aspartic protease enzyme; and
(b): adding a suitable amount of a polypeptide formulation comprising polypeptides longer than 10 amino acids to the sample of step (a), wherein the polypeptides are not milk clotting aspartic protease enzymes and not enzymes that degrade the aspartic protease enzymes, to get a liquid milk clotting aspartic protease enzyme composition, wherein the composition comprises:
   (I): milk clotting aspartic protease enzyme at a strength of from 26 IMCU/g to 30,100 IMCU/g of the composition; and
   (II): the in step (b) added not milk clotting aspartic protease enzyme polypeptides longer than 10 amino acids in a concentration from 0.01% to 10% (w/w) of the composition; and
      wherein the strength (IMCU/g of the composition) in (I) is at least 1% higher than the strength in (i) (IMCU/g of the sample), measured after one week of storage at 5° C.

In a preferred embodiment, the strength (IMCU/g of the composition) in (I) is at least 3% higher than the strength in (i) (IMCU/g of the composition), measured after one week of storage at 5° C.; more preferably the strength (IMCU/g of the composition) in (I) is at least 7% higher than the strength in (i) (IMCU/g of the composition), measured after one week of storage at 5° C.; even more preferably the strength (IMCU/g of the composition) in (I) is at least 10% higher than the strength in (i) (IMCU/g of the composition), measured after one week of storage at 5° C.; and most preferably the strength (IMCU/g of the composition) in (I) is at least 15% higher than the strength in (i) (IMCU/g of the composition), measured after one week of storage at 5° C.

Preferred examples/embodiments of milk clotting aspartic protease enzymes are described above.

Preferred examples/embodiments of “polypeptides longer than 10 amino acids” are described above.

It is preferred that the enzyme strength in item (i) is a strength of from 100 IMCU/g of the sample to 10,000 IMCU/g of the sample, more preferably a strength of from 500 IMCU/g of the sample to 6000 IMCU/g of the sample.

It is preferred that the enzyme strength in item (I) is a strength of from 100 IMCU/g of the composition to 10,000 IMCU/g of the composition, more preferably a strength of from 500 IMCU/g of the composition to 6000 IMCU/g of the composition.

Preferably, the purified sample of step (a)(ii) is a sample, wherein at least 80% of the total amounts of proteins with a size bigger than 10 kDa, determined by SDS-PAGE, in the purified sample are milk clotting aspartic protease enzyme;

more preferably, wherein at least 90% of the total amounts of proteins with a size bigger than 10 kDa, determined by SDS-PAGE, in the purified sample are milk clotting aspartic protease enzyme;

even more preferably wherein at least 95% of the total amounts of proteins with a size bigger than 10 kDa, determined by SDS-PAGE, in the purified sample are milk clotting aspartic protease enzyme.

It may be preferred that at least 99% of the total amounts of proteins with a size bigger than 10 kDa, determined by SDS-PAGE, in the purified sample are milk clotting aspartic protease enzyme.

As known to the skilled person—in the present context it is routine work for the skilled person to obtain purified liquid milk clotting aspartic protease enzyme sample as discussed herein—for instance by use of suitable chromatography (e.g. column chromatography) isolation procedures. As such chromatography is well known to the skilled person are it is therefore not necessary to describe chromatography procedures as such in details herein.

For instance, WO02/36752A2 (Chr. Hansen) describes a recombinant method to produce Camelius dromedarius chymosin (Camel chymosin) using Aspergillus cells (preferably Aspergillus niger) as production host cells.

It is also known to use other cells as production host cells—such as e.g. yeast cell, where an example is e.g. Kluyveromyces cells (for instance Kluyveromyces lactis).

Accordingly, it may be preferred that the purified sample of step (a)(ii) is a sample obtained from recombinant production of the milk clotting aspartic protease enzyme in fungal or yeast production host cells, such as e.g. Aspergillus cells or Kluyveromyces cells.

As known in the art—Mucorpepsin derived from Rhizomucor miehei may preferably be produced by use of Rhizomucor miehei as production host cell.

In relation to item (II)—it is preferred that the in step (b) added not milk clotting aspartic protease enzyme polypeptides longer than 10 amino acids is in a concentration from 0.05% to 8% (w/w) of the composition; more preferably in a concentration from 0.1% to 7% (w/w) of the composition; even more preferably in a concentration from 0.25% to 5% (w/w) of the composition and most preferably in a concentration from 0.5% to 4% (w/w) of the composition (such as e.g. in a concentration from 1% to 3% (w/w) of the composition).

Fifth and/or Sixth Aspect—A Liquid and/or Dried Milk Clotting Aspartic Protease Enzyme Composition

As discussed above—the fifth aspect of the invention relates to a liquid milk clotting aspartic protease enzyme composition comprising:
(I): milk clotting aspartic protease enzyme at a strength of from 25 IMCU/g to 30,000 IMCU/g of the composition;
(II): not milk clotting aspartic protease enzyme polypeptides longer than 10 amino acids in a concentration from 0.01% to 10% (w/w) of the composition; and
(III): a salt in a concentration from 1 to 350 g/kg and wherein the pH of the composition is from 2 to 8; and
   (x): wherein the polypeptides longer than 10 amino acids of item (II) are at least one polypeptide selected from the group of polypeptides consisting of: whey proteins, alpha lactalbumin, beta-lactoglobulin, transferrin, lactoperoxidase, casein, alpha-s1-casein, alpha-s2-casein, beta-casein, kappa-casein, ovalbumin, gelatin, bovine serum albumin, soy proteins, pea proteins, corn proteins, potato proteins, hemp proteins, rice proteins, spirulina proteins, wheat proteins, peanut proteins, sun flower proteins, rape seed proteins, blood proteins and algae proteins.

As discussed above—the sixth aspect of the invention relates to a dried granulated milk clotting aspartic protease enzyme composition comprising:
(I): milk clotting aspartic protease enzyme at a strength of from 25 IMCU/g to 30,000 IMCU/g of the composition;
(II): not milk clotting aspartic protease enzyme polypeptides longer than 10 amino acids in a concentration from 0.01% to 10% (w/w) of the composition; and
(III): a salt and wherein the pH of the composition suspended in water is from 2 to 8; and
   (x): wherein the polypeptides longer than 10 amino acids of item (II) are at least one polypeptide selected from the group of polypeptides consisting of: whey proteins, alpha lactalbumin, beta-lactoglobulin, transferrin, lactoperoxidase, casein, alpha-s1-casein, alpha-s2-casein, beta-casein, kappa-casein, ovalbumin, gelatin, bovine serum albumin, soy proteins, pea proteins, corn proteins, potato proteins, hemp proteins, rice proteins, spirulina proteins, wheat proteins, peanut proteins, sun flower proteins, rape seed proteins, blood proteins and algae proteins.

For both the liquid and the dried composition—preferred examples/embodiments of milk clotting aspartic protease enzymes are described above.

For both the liquid and the dried composition—preferred examples/embodiments of “polypeptides longer than 10 amino acids” are described above.

For both the liquid and the dried composition—it is preferred that the enzyme strength in item (I) is a strength of from 100 IMCU/g of the composition to 10,000 IMCU/g of the composition, more preferably a strength of from 500 IMCU/g of the composition to 6000 IMCU/g of the composition.

For both the liquid and the dried composition—in relation to item (II), it is preferred the not milk clotting aspartic protease enzyme polypeptides longer than 10 amino acids is in a concentration from 0.05% to 8% (w/w) of the composition; more preferably in a concentration from 0.1% to 7% (w/w) of the composition; even more preferably in a concentration from 0.25% to 5% (w/w) of the composition and most preferably in a concentration from 0.5% to 4% (w/w) of the composition (such as e.g. in a concentration from 1% to 3% (w/w) of the composition).

For the liquid composition—the salt in item (iii) is preferably in a concentration from 10 to 300 g/kg, more preferably is in a concentration from 25 to 250 g/kg.

As known to the skilled person—for the dried composition the salt concentration in item (iii) may be relatively high—such as e.g. from 50% (w/w) to 99.9% (w/w) or such as e.g. from 80% (w/w) to 99% (w/w).

For both the liquid and the dried composition—it is preferred that the salt is an inorganic salt—preferably wherein the inorganic salt is selected from the group of NaCl, KCl, Na₂SO₄, (NH₄)₂SO₄, K₂HPO₄, KH₂PO₄, Na₂HPO₄ or NaH₂PO₄ or a combination thereof. Most preferably, the salt is NaCl.

Both the liquid and the dried composition may comprise further additives/compounds such as e.g. a preservative.

As known to the skilled person—preservative may generally be added in a concentration sufficient to prevent microbial growth during shelf life of the product.

Examples of preservatives may be e.g. weak organic acids such as formate, acetate, lactate, propionate, malate, benzoate, sorbate or fumarate. Parabens (alkyl esters of para-hydroxybenzoate) may also be used as preservative. Glycerol or propanediol has also been described as preservatives.

Both the liquid and the dried composition—it is preferred that the pH is from 3 to 7, more preferably that the pH is from 4 to 6.5 and even more preferably that the pH is from 5 to 6.

Preferably, the liquid composition is an aqueous composition, for instance an aqueous solution. As used herein an aqueous composition or aqueous solution encompasses any composition or solution comprising water, for instance at least 20 wt % of water, for instance at least 40 wt % of water.

It may be preferred that the liquid composition as described herein has a total weight of from 10 g to 10,000 kg, such as e.g. from 100 g to 3000 kg.

It may be preferred that the dried granulated composition as described herein has a total weight of from 0.25 g to 200 kg, such as e.g. from 0.5 g to 50 kg.

It is preferred that the composition is a liquid milk clotting aspartic protease enzyme composition as described herein.

For both the liquid and the dried composition—a preferred embodiment is:
(y): wherein the sum of the amounts of aspartic protease enzyme of item (I) and polypeptides longer than 10 amino acids of item (II), determined by SDS-PAGE, are higher than 50% (w/w) of the total amount of proteins and/or polypeptides longer than 10 amino acids in the composition; more preferably
(y): wherein the sum of the amounts of aspartic protease enzyme of item (I) and polypeptides longer than 10 amino acids of item (II), determined by SDS-PAGE, are higher than 70% (w/w) of the total amount of proteins and/or polypeptides longer than 10 amino acids in the composition; even more preferably
(y): wherein the sum of the amounts of aspartic protease enzyme of item (I) and polypeptides longer than 10 amino acids of item (II), determined by SDS-PAGE, are higher than 80% (w/w) of the total amount of proteins and/or polypeptides longer than 10 amino acids in the composition; and most preferably
(y): wherein the sum of the amounts of aspartic protease enzyme of item (I) and polypeptides longer than 10 amino acids of item (II), determined by SDS-PAGE, are higher than 90% (w/w) of the total amount of proteins and/or polypeptides longer than 10 amino acids in the composition.

In the present context—the skilled person will know or may routinely determine (e.g. based on specific relevant amino acid sequences) the origin of the polypeptides longer than 10 amino acids of item (x).

Accordingly, the skilled person may therefore also routine determine (e.g. via SDS-PAGE) if item (y) is fulfilled in a herein relevant composition of interest.

### Seventh Aspect and Eight Aspect

As discussed above—the seventh aspect of the invention relates to a liquid milk clotting aspartic protease enzyme composition comprising milk clotting aspartic protease enzyme at a strength of from 1600 IMCU/g to 30,000 IMCU/g of the composition and a salt in a concentration from 1 to 350 g/kg and wherein the pH of the composition is from 2 to 8.

An eight aspect of the invention relates to a dried granulated milk clotting aspartic protease enzyme composition comprising milk clotting aspartic protease enzyme at a strength of from 1600 IMCU/g to 30,000 IMCU/g of the composition and a salt and wherein the pH of the composition suspended in water is from 2 to 8.

For both the liquid and the dried composition—preferably, the strength is a strength of from 2000 IMCU/g to 15,000 IMCU/g of the composition, such as from 3000 IMCU/g to 10,000 IMCU/g of the composition or such as from 4000 IMCU/g to 8000 IMCU/g of the composition.

### Ninth Aspect—A Method for a Method for Making a Food or Feed Product

As discussed above—a milk clotting aspartic protease enzyme composition as described herein may be used according to the art—e.g. to make a milk based product of interest (such as e.g. a cheese product).

As discussed above—the ninth aspect of the invention relates to a method for making a food or feed product comprising adding an effective amount of a milk clotting aspartic protease enzyme composition of any of fifth to eight aspect or any herein relevant embodiments thereof to the food or feed ingredient(s) and carrying out further manufacturing steps to obtain the food or feed product.

Preferably, the food or feed product is a milk based product and wherein the method comprises adding an effective amount of the isolated chymosin polypeptide variant as described herein to milk and carrying our further manufacturing steps to obtain the milk based product.

The milk may e.g. be sheep milk, goat milk, buffalo milk, yak milk, lama milk, camel milk or cow milk.

The milk based product may e.g. be a fermented milk product, a quark or a cheese.

It may be preferred that the method for making a food or feed product of the fourth aspect or herein relevant embodiments thereof is a method, wherein a milk clotting aspartic protease enzyme composition first have been stored according to the method for storage of a milk clotting aspartic protease enzyme of the third aspect and thereafter added to the food or feed ingredient(s) according to the method for making a food or feed product of the fourth aspect.

### EXAMPLES

### Example 1

### Determination of Specific Milk-Clotting Activity

### 4.1 Determination of Clotting Activity

Milk clotting activity was determined using the REMCAT method, which is the standard method developed by the International Dairy Federation (IDF method).

Milk clotting activity is determined from the time needed for a visible flocculation of a standard milk substrate prepared from a low-heat, low fat milk powder with a calcium chloride solution of 0.5 g per litre (pH≈6.5). The clotting time of a milk-clotting enzyme sample is compared to that of a reference standard having known milk-clotting activity and having the same enzyme composition by IDF Standard 110B as the sample. Samples and reference standards were measured under identical chemical and physical conditions. Variant samples were adjusted to approximately 3 IMCU/ml using an 84 mM acetic acid pH 5.5 buffer. Hereafter, 200 μl enzyme was added to 10 ml preheated milk (32° C.) in a glass test tube placed in a water bath, capable of maintaining a constant temperature of 32° C.±1° C. under constant stirring.

The total milk-clotting activity (strength) of a milk-clotting enzyme is calculated in International Milk-Clotting Units (IMCU) per ml relative to a standard having the same enzyme composition as the sample according to the formula:

$$Strength in IMCU \text{/} ml = \frac{Sstandard \times Tstandard \times Dsample}{Dstandard \times Tsample}$$

Sstandard: The milk-clotting activity of the international reference standard for rennet.
Tstandard: Clotting time in seconds obtained for the standard dilution.
Dsample: Dilution factor for the sample
Dstandard: Dilution factor for the standard
Tsample: Clotting time in seconds obtained for the diluted rennet sample from addition of enzyme to time of flocculation

### 4.2 Determination of Total Protein Content

Total protein content was determined using the Pierce BCA Protein Assay Kit from Thermo Scientific following the instructions of the providers.

### 4.3 Calculation of Specific Clotting Activity

Specific clotting activity (IMCU/mg total protein) was determined by dividing the clotting activity (IMCU/ml) by the total protein content (mg total protein per ml).

### Example 2

### Enzyme Preparations

Bovine chymosin (CHY-MAX®, Chr. Hansen A/S) or camel chymosin (CHY-MAX® M, Chr. Hansen A/S) were recombinantly expressed in Aspergillus niger (roughly as described in WO02/36752A2). The enzymes were purified by chromatography technology. Mucorpepsin (Hannilase®, Chr. Hansen A/S) derived from Rhizomucor miehei was produced by use of Rhizomucor miehei as production host cell and purified by chromatography technology. For all the purified enzyme samples—at least 90% of the total amounts of proteins with a size bigger than 10 kDa, determined by SDS-PAGE, in the purified sample were the relevant milk clotting aspartic protease enzymes.

All enzyme samples were prepared by mixing an exact volume for a stock solution of the enzyme with a solution of the additive and adding buffer to a final volume. In this manner the concentration of enzyme protein is kept constant for all prepared samples. Buffer composition was: 0.25 M sodium acetate, 20 mM sodium phosphate, 2.0 M sodium chloride, pH 5.7, 5 mM methionine, and 35 mM sodium benzoate. The strength was from 200 to 1200 IMCU/ml. The composition was added different polypeptides such as Hammersten casein, WPC80, Lacprodan Alpha 10, Lacprodan Alpha 20, etc.

WPC80 is a commercial preparation of dried whey protein concentrate with 80% protein. Lacprodan Alpha 10 and Lacprodan Alpha 20 are commercial preparations of whey protein isolate containing 43% and 60% alpha lactalbumin of total protein content, respectively.

### Example 3

### Physical Stability of Camel Chymosin

Liquid formulations of industrial enzymes are subjected to physical forces from unit operations such as pumping, stirring and filtration over membranes. During transportation of partly filled containers sloshing around of liquid formulation may also contribute to this. Shear stress and increased exposure of enzyme to the water-air interface may induce denaturation and concomitant loss of enzyme activity.

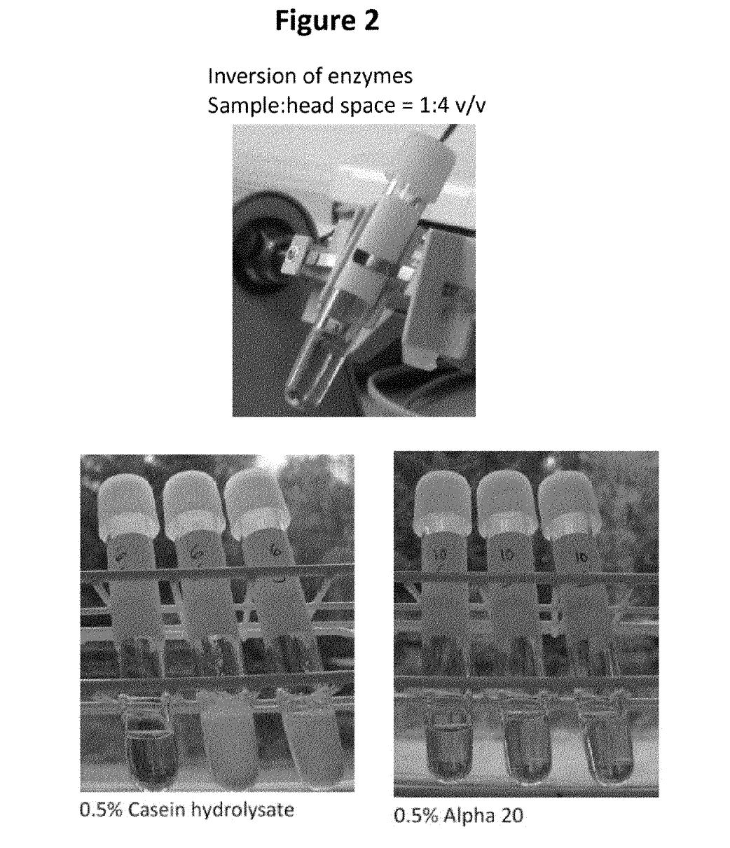

Physical stability of an enzyme or protein sample can be tested by repeatable shaking a sample of the enzyme in a test tube having high head space to sample volume ratio. The stability of different aspartic proteases towards shaking was investigated by inverting a 2 ml sample filled in a 10 ml tube in a rotary device for 1 hour (see FIG. 2 herein). For each solution, relative milk clotting activity was measured after 1 hour of vertical inversion (at room temperature) and compared to a non-inverted control having the exact same composition. Results were expressed as “retained activity” which is obtained by diving activity of the inverted sample with the activity of the non-inverted control sample.

### Results:

Vertical inversion for 1 hr of a sample of camel chymosin results in an activity loss of more than 30% cf. Table 1 (No addition). Addition of PEG8000 to a concentration of 0.015% was found to protect camel chymosin and resulting in no loss of activity upon vertical inversion. When the sample of camel chymosin contained Hammersten casein, Alpha 10, Alpha 20 or WPC 80 at a concentration of either 0.5% w/v or 1.0% w/v practically no loss upon vertical inversion was seen.

The protecting effect of Alpha 20 and WPC 80 was found to decrease gradually when their concentration was decreased below 0.1%. A formulation of camel chymosin with either acid casein hydrolysate or whey permeate did not increase stability of the enzyme as the loss in activity upon vertical inversion was the same as for the untreated control sample (Table 3).

Conclusion: The results show that certain proteins added to a preparation of camel chymosin can increase the physical stability of the enzyme.

TABLE 1
Retained activity of camel chymosin samples subjected to vertical
inversion for 1 hr (Internal ref number: EXP-13-AD2681).

| Compound | Retained activity |
|---|---|
| No addition | 67% (3%) |
| PEG (0.015%) | 99% (0%) |
| Glycerol (6%) | 67% (4%) |
| 0.5% Hammersten casein | 97% (0%) |
| 1% Hammersten casein | 97% (1%) |
| 0.5% Casein hydrolysate (Merck) | 67% (4%) |
| 1% Casein hydrolysate (Merck) | 74% (5%) |
| 0.5% Alpha 10 (Prøve 1) | 98% (0%) |
| 1% Alpha 10 (Prøve 1) | 98% (0%) |
| 0.5% Alpha 20 (Prøve 2) | 98% (0%) |
| 1% Alpha 20 | 98% (0%) |
| 0.5% Permeat | 64% (17%) |
| 1% Permeat | 69% (1%) |
| 0.1% WPC 80 | 100% (0%) |
| 1% WPC 80 | 99% (0%) |

### Example 4

### Specific Activity of Camel Chymosin

### Results:

The inversion experiments were designed so the exact same amount of enzyme protein was added in each experiment and all formulations were made up to the same volume. If composition of the formulation did not influence enzymatic activity, one would expect to find the same enzymatic activity of all control samples, i.e. samples not inverted. However, this was not the case. Samples containing PEG8000 were 4-5% higher in activity in good accordance with recently submitted patent application IN5103DK00. Samples containing Hammerstein casein, Alpha 20 or WPC 80 had 13-18% higher activity compared to the sample without additives (no addition) even though the same amount of enzyme protein was applied (Table 2). This shows that the presence milk proteins in the formulation of camel chymosin increase the specific activity of the enzyme. The same conclusion is made from Table 3 which shows the activity of compositions containing Alpha 20 and WPC80 at five different concentrations. Table 5 show activity of the composition, activity index with ‘no addition’=100%, and the retained activity of a sample subjected to vertical inversion for 1 hr.

Addition of polypeptides to a composition of camel chymosin has two effects on the enzyme: Increase in specific activity and an increased physical stability of the enzyme. Both properties correlate well with the dosage of polypeptide as seen from Table 3. In Table 3 it is found that when the concentration of Alpha 20 is decreased from 0.5 to 0.01%, the enzyme activity drops from index 115 to index 102 which is almost the same level as the untreated control. At a concentration of 0.01% Alpha 20 the physical stability (retained activity) of the enzyme is the same as in the control experiment (no addition).

TABLE 2
Activity of CHY-MAX ® M added different polypeptides.
Activity index relative to untreated control (‘no addition’)
is shown in percentage (Internal ref number: EXP-13-AD2681).

| Compound | Activity (IMCU/ml) | |
|---|---|---|
| No addition | 1124 (13.3) | 100% |
| PEG (0.015%) | 1163 (6.8) | 104% |
| Glycerol (6%) | 1069 (12.0) | 95% |
| 0.5% Hammersten casein | 1251 (12.3) | 111% |
| 1% Hammersten casein | 1275 (25.0) | 113% |
| 0.5% Casein hydrolysate (Merck) | 1112 (12.3) | 99% |
| 1% Casein hydrolysate (Merck) | 1102 (19.8) | 98% |
| 0.5% Alpha 10 (Prøve 1) | 1282 (8.6) | 114% |
| 1% Alpha 10 (Prøve 1) | 1305 (9.9) | 116% |
| 0.5% Alpha 20 (Prøve 2) | 1287 (5.0) | 115% |
| 1% Alpha 20 (Prøve 2) | 1323 (1.2) | 118% |
| 0.5% Permeat (Prøve 3) | 1147 (15.6) | 102% |
| 1% Permeat (Prøve 3) | 1127 (32.2) | 100% |
| 0.1% WPC 80 (Prøve 4) | 1248 (15.3) | 111% |
| 1% WPC 80 (Prøve 4) | 1287 (2.8) | 115% |

TABLE 3
Activity of CHY-MAX ® M added different polypeptides.
Activity index relative to untreated control (‘no addition’)
is shown in percentage. Column to the right show retained
activity of camel chymosin samples subjected to vertical
inversion for 1 hr (Internal ref number: EXP-13-AD2681).

| | | |
|---|---|---|
| Compound | Activity (IMCU/ml) | Retained activity |

| | | | |
|---|---|---|---|
| No addition | 1102 (19.6) | 100% | 76% (4%) |
| PEG, 0.015% | 1153 (12.4) | 105% | 101% (2%) |
| Alpha 20, 0.5% | 1269 (13.0) | 115% | 100% (1%) |
| Alpha 20, 0.25% | 1224 (8.5) | 111% | 100% (1%) |
| Alpha 20, 0.1 | 1162 (1.2) | 105% | 100% (0%) |
| Alpha 20, 0.05% | 1134 (7.2) | 103% | 97% (0%) |
| Alpha 20, 0.01% | 1129 (1.0) | 102% | 79% (0%) |
| WPC 80, 0.5% | 1235 (13.9) | 112% | 101% (1%) |
| WPC 80, 0.25% | 1227 (10.2) | 111% | 98% (0%) |
| WPC 80, 0.1% | 1155 (14.3) | 105% | 100% (1%) |
| WPC 80, 0.05% | 1141 (7.3) | 104% | 95% (2%) |
| WPC 80, 0.01% | 1112 (5.4) | 101% | 80% (1%) |

Conclusion: Addition of polypeptides to a composition of camel chymosin has two effects on the enzyme: Increase in specific activity and an increased physical stability of the enzyme. Both properties correlate well with the dosage of polypeptide.

### Example 5

### Specific Activity of Camel Chymosin, Bovine Chymosin, Mucor Pepsin L and Mucor Pepsin XL

TABLE 4
Activity of milk clotting enzymes added different polypeptides (Internal ref
number: EXP-13-AD2690). Standard deviation in absolute numbers is in parenthesis.

| Compound | CHY-MAX ® M | | CHY-MAX ® | | Hannilase L | | Hannilase XP | |
|---|---|---|---|---|---|---|---|---|
| Control | 1066 (18.7) | 100% | 1117 (11.7) | 100% | 985 (4.0) | 100% | 1041 (2.3) | 100% |
| Alpha 20, | 1108 (6.2) | 104% | 1143 (2.7) | 102% | 983 (9.4) | 99% | 1035 (7.1) | 100% |
| 0.1% | | | | | | | | |
| Alpha 20, | 1192 (20.1) | 111% | 1173 (3.3) | 105% | 994 (7.6) | 100% | 1044 (2.3) | 100% |
| 0.5% | | | | | | | | |
| Alpha 20, | 1225 (6.8) | 116% | 1191 (3.0) | 107% | 1,007 (11.0) | 101% | 1057 (5.1) | 102% |
| 1.0% | | | | | | | | |
| WPC80 0.1% | 1086 (11.4) | 103% | 1131 (9.1) | 101% | 980 (10.2) | 101% | 1030 (13.5) | 100% |
| WPC80 0.5% | 1156 (11.0) | 110% | 1151 (7.2) | 102% | 995 (9.8) | 101% | 1034 (2.5) | 99% |
| WPC80 1.0% | 1196 (20.5) | 114% | 1170 (15.6) | 104% | 997 (3.7) | 102% | 1047 (9.7) | 101% |

Conclusion: The results in table 2-4 show that addition of polypeptides to a formulation of aspartic proteases increase the specific activity.

### Example 6

The table below show results from tests performed similar to Example 3 above—but using different protein formulations. All proteins shown in the table were added to a final content of 1% w/w and with gliadin as only exception gave clear solutions. Physical stability was tested according to example 3. Samples were stored for 1 year at 5° C. and 37° C., respectively, and stability was tested during storage period. The column ‘End of storage’ shows remaining activity after 1 year—the number was calculated by fitting a single exponential function to all data points.

As known in the art—the term “peptone” refers to proteins digested by proteolysis.

As known in the art—the term “tryptone” refers to proteins digested by the protease trypsin.

TABLE 5
Activity of CHY-MAX M added different polypeptides at 1% w/w. Activity index relative
to untreated control (‘no addition’) is shown in percentage. Column ‘Retained
Activity’ show retained activity of camel chymosin samples subjected to vertical
inversion for 1 hr. Column ‘End of Storage’ show activity after storage at 5°
C. and 37° C. Standard deviation is shown in parenthesis. (Internal ref number: EXP-14-AE8307)

| | | Activity | | Retained | End of storage |
|---|---|---|---|---|---|
| | Compound | IMCU/ml/Index | | activity | (5° C./37° C.) |
| 1 | Control (no addition) | 389 (3.8) | 100% | 36% (2.4) | 101%/14% |
| 2 | 18332 Peptone (vegetable) | 400 (11.1) | 106% | 95% (3.8) | 91%/13% |
| 3 | 51841 Peptone (vegetable) acid | 418 (1.5) | 109% | 99% (4.6) | 94%/13% |
| | hydrolysate | | | | |
| 4 | 19942 Peptone (vegetable), no 1 | 422 (5.5) | 108% | 97% (0.6) | 93%/15% |
| 5 | 61854 Peptone (vegetable), no 2 | 423 (3.7) | 109% | 99% (1.4) | 93%/13% |
| 6 | 92976 Peptone special (vegetable) | 423 (1.8) | 110% | 93% (7.1) | 93%/13% |
| 7 | 29185 Proteose Peptone (vegetable) | 428 (3.0) | 110% | 97% (0.6) | 94%/12% |
| 8 | 16922 Tryptone (vegetable) | 431 (2.9) | 111% | 96% (0.6) | 93%/13% |
| 9 | 12331 Tryptose (vegetable) | 428 (2.0) | 111% | 94% (5) | 96%/15% |
| 10 | 05138 Vegetable Extract | 412 (9.8) | 109% | 98% (2.3) | 100%/14% |
| 11 | 04316 Vegetable Extract no 1 | 414 (3.1) | 108% | 97% (5.1) | 97%/15% |
| 12 | 49869 Vegetable Extract no 2 | 409 (2.3) | 106% | 99% (3.5) | 97%/15% |
| 13 | 07436 Vegetable hydrolysate no 2 | 411 (5.8) | 107% | 101% (2.5) | 98%/14% |
| 14 | 67381 Vegetable Infusion powder | 403 (5.9) | 103% | 99% (2.9) | 102%/13% |
| 15 | 95757 Vegetable Special Infusion | 411 (1.6) | 106% | 98% (6.4) | 99%/18% |
| | powder | | | | |
| 16 | 83059 Peptone from potatoes | 403 (1.9) | 104% | 99% (1.5) | 100%/13% |
| 17 | 93491 Peptone from broad bean | 425 (10.2) | 108% | 99% (1.4) | 96%/12% |
| 18 | 93492 Peptone from wheat | 427 (1.2) | 110% | 100% (2.2) | 100%/13% |
| 19 | 90765 Peptone from soybean, | 408 (11.2) | 108% | 100% (2.3) | 102%/18% |
| | enzymatic digest | | | | |
| 20 | 70178 Peptone from soybean, | 401 (0.3) | 104% | 92% (2.3) | 98%/14% |
| | enzymatic digest | | | | |
| 21 | S1674 Soy protein acid hydrolysate | 432 (13.7) | 109% | 88% (1.3) | 106%/18% |
| 22 | 87972 Peptone from soybean, | 400 (7.0) | 102% | 91% (0.6) | 106%/21% |
| | enzymatic digest | | | | |
| 23 | 96174 Peptone from pea | 404 (2.9) | 104% | 98% (1.3) | 98%/10% |
| 25 | 49760 GLUTEN HYDROLYSATE F. | 432 (5.1) | 111% | 95% (2) | 107%/16% |
| | MAIZE | | | | |
| 26 | Ovalbumin | 464 (4.9) | 121% | 99% (1.1) | 97%/14% |
| 27 | Gelatin | 475 (6.0) | 124% | 99% (1.7) | 101%/16% |
| 28 | Bovine serum albumin | 487 (8.1) | 128% | 100% (2.6) | 104%/15% |
| 29 | PEG8000 (0.015%) | 443 (1.8) | 115% | 98% (0.1) | 104%/15% |

### Example 7

Extracts of plant proteins were prepared by suspending 2 g sample in 40 ml brine consisting of: 12% NaCl, 20 g/L NaAc anhydrous, 2.5 g/L NaH2PO4 anhydrous, and 10 g/L Na-benzoate in water, pH 5.4-5.8. After mixing for 2 hours on rotating device the suspension was centrifuged and the supernatant pH adjusted to 5.4-5.8 and filtered through a 0.45 μm syringe filter. The extracts were used for preparing formulations of CHY-MAX M by mixing with an exact measured and equal volume of a stock solution the enzyme to give samples having same concentration of enzyme proteins. In this example, extracts of 27 different plant proteins were tested in three groups with each group prepared on different days.

The activity was measured one day after sample preparation; the column titled activity shown activity in IMCU/ml and relative to untreated control (no addition). Physical stability was tested according to example 3 with the only difference that in present example a different rotary device was used for vertical inversion of the samples (Multi RS-60 from Biosan at 32 rpm for 1 hour). The change in rotary device may have resulted in increased physical stress of the samples since retained activity of untreated sample was only 10% compared to ca. 70% in preceding examples. Samples were stored for 1 year at 5° C. and 37° C., respectively, and stability was tested during storage period. The column ‘End of storage’ shows remaining activity after 1 year—the number was calculated by fitting a single exponential function to all data points.

TABLE 6
Activity of CHY-MAX ® M added different polypeptides. Activity
index relative to untreated control (‘no addition’) is shown
in percentage. Column ‘Retained Activity’ show retained activity
of camel chymosin samples subjected to vertical inversion for 1
hr. Column ‘End of Storage’ show activity after storage at
5° C. and 37° C. Standard deviation is shown in parenthesis.
Internal ref number: Group 1/EXP-14-AF3604.

| | | Activity | | Retained | End of storage |
|---|---|---|---|---|---|
| | Compound | IMCU/ml/Index | | activity | (5° C./37° C.) |
| | No addition (only Brine) | 405 (6.8) | 100% | 10% (1.4) | 96%/22% |
| | 0.015% PEG 8000 | 481.9 (6.5) | 119% | 99% (3.1) | 95%/21% |
| 1 | Nutralys F 85 F | 450.4 (6) | 111% | 96% (1) | 87%/17% |
| | (Roquette) | | | | |
| 2 | Nutralys F85G | 421.6 (4.6) | 104% | 102% (0.5) | 91%/20% |
| | (Roquette) | | | | |
| 3 | Nutralys F 85 M | 419.9 (13.8) | 104% | 102% (0.9) | 89%/10% |
| | (Roquette) | | | | |
| 4 | Nutralys S 85 F | 415.2 (6.2) | 103% | 102% (0.2) | 87%/19% |
| | (Roquette) | | | | |
| 5 | Nutralys W | 461.3 (11.3) | 114% | 100% (0.4) | 87%/14% |
| | (Roquette) | | | | |
| 6 | Solulys 048E | 399.5 (12.4) | 99% | 103% (1) | 94%/8% |
| | (Roquette) | | | | |
| 7 | Solulys 095E | 412.3 (2.6) | 102% | 100% (1.8) | 91%/9% |
| | (Roquette) | | | | |
| 8 | Tubermine Fv Proteine | 403.4 (5.7) | 100% | 96% (0) | 94%/15% |
| | De Pomme Des Terre | | | | |
| | (Roquette) | | | | |
| 9 | Tubermine GP Proteine | 411 (3.3) | 101% | 90% (0.7) | 95%/17% |
| | De Pomme Des Terre | | | | |
| | (Roquette) | | | | |

TABLE 7
Activity of CHY-MAX ®M added different polypeptides. Activity index
relative to untreated control (‘no addition’) is shown in percentage. Column
‘Retained Activity’ show retained activity of camel chymosin samples
subjected to vertical inversion for 1 hr. Column ‘End of Storage’ show
activity after storage at 5° C. and 37° C. Standard deviation is
shown in parenthesis. Internal ref number: Group 2/EXP-14-AF3604.

| | | Activity | | Retained | End of storage |
|---|---|---|---|---|---|
| | Compound | IMCU/ml/Index | | activity | (5° C./37° C.) |
| | No addition (only Brine) | 395.9 (10.8) | 100% | 7% (1.25) | 93%/24% |
| | No addition (only Brine) | 385 (3.7) | 97% | 7.5% (0.49) | — |
| | No addition (only Brine) | 396.3 (4.9) | 100% | 6.3% (0.54) | — |
| | 0.015% PEG 8000 | 457.8 (5.6) | 116% | 98.9% (1.13) | 99%/24% |
| 10 | Alburex E 361 G | 511.1 (1) | 129% | 92.7% (0.84) | 95%/21% |
| | (Roquette) | | | | |
| 11 | Gluten De Ble Supra | 423.2 (2.2) | 107% | 90% (2.83) | 92%/17% |
| | Vital (Roquette) | | | | |
| 12 | Lysamine GP Proteine | 391.4 (13.6) | 99% | 77.3% (0.88) | 95%/14% |
| | De Pois (Roquette) | | | | |
| 13 | Corn Gluten | 359.1 (18.7) | 91% | 97.3% (0.28) | 103%/22% |
| | (Roquette) | | | | |
| 14 | [symbol] rteprotein | 440.4 (3.8) | 111% | 98.3% (1.36) | 86%/19% |
| | (Naturdrogeriet A/S) | | | | |
| 15 | Sojaprotein | 427 (1.1) | 108% | 106.8% (6.59) | 88%/17% |
| | (Naturdrogeriet A/S) | | | | |
| 16 | Vegan Ris protein | 352.4 (4.3) | 89% | 96.5% (2.22) | 114%/28% |
| | (Naturdrogeriet A/S) | | | | |
| 17 | Sojaprotein | 437 (6) | 110% | 94.2% (2.41) | 89%/18% |
| | Ketolyse A/S | | | | |
| 18 | Hamp protein complex | 422.7 (3.2) | 107% | 98.2% (2.66) | 100%/20% |
| | økologisk (Nybogaard A/S) | | | | |

TABLE 8
Activity of CHY-MAX ® M added different polypeptides. Activity index
relative to untreated control (‘no addition’) is shown in percentage. Column
‘Retained Activity’ show retained activity of camel chymosin samples
subjected to vertical inversion for 1 hr. Column ‘End of Storage’ show
activity after storage at 5° C. and 37° C. Standard deviation is
shown in parenthesis. Internal ref number: Group 3/EXP-14-AF3604.

| | | Activity | | Retained | End of storage |
|---|---|---|---|---|---|
| | Compound | IMCU/ml/Index | | activity | (5° C./37° C.) |
| | No addition (only Brine) | 419.5 (1.1) | 100% | 8.8% (2.38) | 97%/28% |
| | 0.015% PEG 8000 | 478.1 (2.9) | 114% | 102.8% (1.12) | 100%/29% |
| 19 | Spirulina pulver økologisk | 484.8 (9.4) | 116% | 97% (0.17) | 57%/1% |
| | (Dinsundhed.Net Aps) | | | | |
| 20 | Plant force rice protein | 413.4 (5) | 99% | 87.5% (1.45) | 96%/17% |
| | (Third Wave Nutrition) | | | | |
| 21 | Superfruict Hemp protein | 447.8 (4.1) | 107% | 97.7% (1.81) | 97%/20% |
| | (Superfruit Scandinavia AB) | | | | |
| 22 | Hampe protein økologisk | 421.2 (1.3) | 100% | 101.2% (2.12) | 98%/23% |
| | (Nybogaard A/S) | | | | |
| 23 | Plant force Rice protein | 412.7 (9.8) | 98% | 84.1% (5.77) | 99%/18% |
| | (Third Wave Nutrition) | | | | |
| 24 | Nutralys T 65 M | 454.5 (8.5) | 108% | 100.1% (0.34) | 88%/14% |
| | (Roquette) | | | | |
| 25 | LAB 4460 Nutralys PEA | 451.9 (13.1) | 108% | 97.2% (0.1) | 90%/19% |
| | XF EXP (Roquette) | | | | |
| 26 | LAB 4462 Nutralys PEA | 436.8 (10.5) | 104% | 99.9% (0.45) | 93%/16% |
| | BF EXP (Roquette) | | | | |
| 27 | Gluten De MAIS 58E | 402.9 (0.5) | 96% | 65.5% (1.33) | 97%/20% |
| | (Roquette) | | | | |

### REFERENCES

1: EP2333056A1 (DSM, date of filing Dec. 4, 2007)
2: WO2012/127005A1 (DSM)
3: DE1492060A1 (Nordmark-Werke GmbH, published in 1969)

1-16. (canceled)

17. A method for making a liquid milk clotting aspartic protease enzyme composition, comprising:
obtaining a liquid milk clotting aspartic protease enzyme sample having a milk clotting aspartic protease enzyme strength of from 25 IMCU/g to 30,000 IMCU/g, wherein milk clotting aspartic protease enzyme constitutes at least 70% by weight of the total amount of proteins present in the sample having a size bigger than 10 kDa as determined by SDS-PAGE; and
adding to the sample an amount of a polypeptide formulation comprising polypeptides longer than 10 amino acid residues, wherein the polypeptides are not milk clotting aspartic protease enzymes and are not enzymes that degrade the milk clotting aspartic protease enzymes,
wherein the amount of added polypeptide formulation results in a concentration of said polypeptides in the composition of from 0.01% to 10% (w/w) and is effective to obtain a liquid milk clotting aspartic protease enzyme composition having a milk clotting aspartic protease enzyme strength of from 26 IMCU/g to 30,100 IMCU/g of the composition, wherein the milk clotting aspartic protease enzyme strength of the composition is at least 1% higher than the milk clotting aspartic protease enzyme strength of the sample when measured after storage for one week at 5° C.

18. The method of claim 17, wherein the milk clotting aspartic protease enzyme comprises chymosin (EC 3.4.23.4).

19. The method of claim 17, wherein the milk clotting aspartic protease enzyme comprises Camelius dromedarius chymosin comprising the “Camel_chymosin” amino acid sequence shown in FIG. 5 or a variant thereof comprising an amino acid sequence having at least 90% sequence identity thereto.

20. The method of claim 17, wherein the milk clotting aspartic protease enzyme comprises bovine pepsin comprising the “Cow_pepsin” amino acid sequence shown in FIG. 5 or a variant thereof comprising an amino acid sequence having at least 90% sequence identity thereto.

21. The method of claim 17, wherein said polypeptides are proteins longer than 75 amino acid residues.

22. The method of claim 17, wherein said polypeptides comprise one or more selected from whey proteins, alpha lactalbumin, beta-lactoglobulin, transferrin, lactoperoxidase, casein, alpha-s1-casein, alpha-s2-casein, beta-casein, kappa-casein, ovalbumin, gelatin, bovine serum albumin, soy proteins, pea proteins, corn proteins, potato proteins, hemp proteins, rice proteins, spirulina proteins, wheat proteins, peanut proteins, sun flower proteins, rape seed proteins, blood proteins, and algae proteins.

23. The method of claim 22, wherein said polypeptides comprise one or more selected from whey proteins, alpha lactalbumin, beta-lactoglobulin, transferrin, lactoperoxidase, casein, alpha-s1-casein, alpha-s2-casein, beta-casein, kappa-casein, ovalbumin, gelatin, and bovine serum albumin.

24. The method of claim 17, wherein the milk clotting aspartic protease enzyme strength of the composition is at least 10% higher than the milk clotting aspartic protease enzyme strength of the sample when measured after storage for one week at 5° C.

25. The method of claim 17, wherein the amount of added polypeptide formulation results in a concentration of said polypeptides in the composition of from 0.5% to 4% (w/w) of the composition.

26. A liquid milk clotting aspartic protease enzyme composition obtained by a method according to claim 17.

27. The method of claim 17, further comprising drying and granulating the composition to obtain a dried granulated milk clotting aspartic protease enzyme composition.

28. A dried granulated milk clotting aspartic protease enzyme composition obtained by a method according to claim 27.

29. A liquid milk clotting aspartic protease enzyme composition, comprising:
(a) milk clotting aspartic protease enzyme at a strength of from 25 IMCU/g to 30,000 IMCU/g of the composition;
(b) polypeptides longer than 10 amino acid residues at a concentration from 0.01% to 10% (w/w) of the composition, wherein the polypeptides are not milk clotting aspartic protease enzymes and comprise one or more selected from whey proteins, alpha lactalbumin, beta-lactoglobulin, transferrin, lactoperoxidase, casein, alpha-s1-casein, alpha-s2-casein, beta-casein, kappa-casein, ovalbumin, gelatin, bovine serum albumin, soy proteins, pea proteins, corn proteins, potato proteins, hemp proteins, rice proteins, spirulina proteins, wheat proteins, peanut proteins, sun flower proteins, rape seed proteins, blood proteins, and algae proteins; and
(c) a salt in a concentration of from 1 to 350 g/kg,
wherein the pH of the composition is from 2 to 8.

30. A liquid milk clotting aspartic protease enzyme composition of claim 29, wherein the milk clotting aspartic protease enzyme comprises chymosin (EC 3.4.23.4).

31. A liquid milk clotting aspartic protease enzyme composition of claim 29, wherein:
(i) said polypeptides comprise one or more selected from whey proteins, alpha lactalbumin, beta-lactoglobulin, transferrin, lactoperoxidase, casein, alpha-s1-casein, alpha-s2-casein, beta-casein, kappa-casein, ovalbumin, gelatin and bovine serum albumin,
(ii) the concentration of said polypeptides is from 0.5% to 4% (w/w) of the composition,
(iii) the sum of the amounts of milk clotting aspartic protease enzyme and said polypeptides, determined by SDS-PAGE, is greater than 50% (w/w) of the total amount of proteins and/or polypeptides longer than 10 amino acid residues present in the composition; and
(iv) the total weight of the composition is from 100 g to 3000 kg.

32. A method for making a food or feed product comprising adding an effective amount of a milk clotting aspartic protease enzyme composition of claim 29 to the food or feed ingredient(s).

33. A dried granulated milk clotting aspartic protease enzyme composition comprising:
(a) milk clotting aspartic protease enzyme at a strength of from 25 IMCU/g to 30,000 IMCU/g of the composition;
(b) polypeptides longer than 10 amino acid residues at a concentration from 0.01% to 10% (w/w) of the composition, wherein the polypeptides are not milk clotting aspartic protease enzymes and comprise one or more selected from whey proteins, alpha lactalbumin, beta-lactoglobulin, transferrin, lactoperoxidase, casein, alpha-s1-casein, alpha-s2-casein, beta-casein, kappa-casein, ovalbumin, gelatin, bovine serum albumin, soy proteins, pea proteins, corn proteins, potato proteins, hemp proteins, rice proteins, spirulina proteins, wheat proteins, peanut proteins, sun flower proteins, rape seed proteins, blood proteins, and algae proteins; and
(c) a salt,
wherein the pH of the composition when suspended in water is from 2 to 8.

34. A dried milk clotting aspartic protease enzyme composition of claim 33, wherein the milk clotting aspartic protease enzyme comprises chymosin (EC 3.4.23.4).

35. A dried milk clotting aspartic protease enzyme composition of claim 33, wherein:
(i) said polypeptides comprise one or more selected from whey proteins, alpha lactalbumin, beta-lactoglobulin, transferrin, lactoperoxidase, casein, alpha-s1-casein, alpha-s2-casein, beta-casein, kappa-casein, ovalbumin, gelatin and bovine serum albumin,
(ii) the concentration of said polypeptides is from 0.5% to 4% (w/w) of the composition,
(iii) the sum of the amounts of milk clotting aspartic protease enzyme and said polypeptides, determined by SDS-PAGE, is greater than 50% (w/w) of the total amount of proteins and/or polypeptides longer than 10 amino acid residues present in the composition; and
(iv) the total weight of the composition is from 0.5 to 50 kg.

36. A method for making a food or feed product comprising adding an effective amount of a milk clotting aspartic protease enzyme composition of claim 34 to the food or feed ingredient(s).
